(19) United States
(12) Patent Application Publication
(10) Pub. No.: US 2026/0331241 A1
(43) Pub. Date: Oct. 22, 2026

(54) COMPUTERIZED SYSTEM AND METHOD FOR ENCODING, VALIDATING, PRICING, AND ELECTRONICALLY PROCESSING NON-ALLOPATHIC HEALTHCARE INTERVENTIONS USING HIPAA-COMPLIANT EDI TRANSACTIONS

(71) Applicant: Jo Melinna SPILLMAN, Carlsbad, NM (US)

(72) Inventor: Jo Melinna SPILLMAN, Carlsbad, NM (US)

(73) Assignee: ABC Coding Solutions, Inc., Boulder, CO (US)

(21) Appl. No.: 19/651,008

(22) Filed: Apr. 17, 2026

Related U.S. Application Data
(60) Provisional application No. 63/792,216, filed on Apr. 21, 2025.

(51) Int. Cl. G16H 10/60; G06Q 30/018; G06Q 40/08

(52) U.S. Cl. CPC G16H 10/60; G06Q 30/018; G06Q 40/084

(57) ABSTRACT

A computerized method and system for encoding, validating, pricing, and mapping healthcare interventions delivered by non-allopathic providers to facilitate electronic transactions among payers, managed care organizations, government programs, and providers using Health Insurance Portability and Accountability Act (HIPAA)-compliant Electronic Data Interchange (EDI) standards. The system integrates ABC codes, practitioner Scope-of-Practice validation logic, Relative Value Unit (RVU)-based procedure pricing, and Universal Product Code (UPC)-based supply pricing. An artificial intelligence intake and normalization layer processes structured data, clinical notes, audio transcriptions, and encounter records to identify interventions, assign codes, validate provider authorization based on jurisdiction-specific rules, and flag incomplete or uncertain data for review—mitigating fraud and expanding access. Validated codes are mapped to standardized administrative code sets and transmitted using ANSI ASC X12N 837 and 835 transaction formats, with 275 Claim Attachments for supplemental details per HIPAA 6020 standards.

### BACKGROUND OF THE INVENTION

### Field of the Invention

The present invention relates to healthcare informatics, and more specifically to computerized methods and systems for encoding, validating, pricing, and electronically processing healthcare interventions delivered by non-allopathic providers. The invention integrates such interventions into the United States healthcare reimbursement infrastructure using Electronic Data Interchange (EDI) standards adopted under the Health Insurance Portability and Accountability Act (HIPAA), 42 U.S.C. § 1320d et seq.

### Description of Related Art

### The Non-Allopathic Provider Gap

Non-allopathic healthcare providers—including but not limited to nurse practitioners, naturopathic doctors, certified nurse-midwives, chiropractors, alternative medicine or holistic physicians, functional medicine practitioners, licensed professional counselors, acupuncturists, nurse anesthetists, nurse midwives, massage therapists, occupational therapists, physical therapists, behavioral health providers, pharmacists, and spiritual care providers—are licensed across all fifty (50) United States and U.S. territories to provide patient care, without physician oversight. According to the National Council for Workforce Analysts (2025), there were 839,108 physicians in the United States treating patients, while more than two (2) million non-allopathic providers operate in private practice settings. And physicians who don't strictly practice allopathic medicine are estimated to number about 200,000. Yet, they also lack coding to describe non-allopathic care. Despite their numerical majority and their substantial contribution to the U.S. healthcare system, non-allopathic providers lack administrative and business parity with conventional physicians when filing healthcare claims for patient reimbursement.

### Historical Development of U.S. Procedural Coding Infrastructure

Since the 1960s, the primary procedural coding infrastructure in the United States has been the Current Procedural Terminology (CPT) system, developed and maintained by the American Medical Association (AMA). In 1983, the U.S. Department of Health and Human Services (HHS) entered into an agreement with the AMA designating CPT as the primary coding system for Medicare, Medicaid, and other federal health programs, simultaneously agreeing to retire any government-maintained codes duplicating CPT codes. This agreement has materially constrained the development of coding infrastructure for non-allopathic interventions.

In 2000, the federal government required state Medicaid agencies to retire over 30,000 state-specific procedure codes. CPT became HCPCS Level I. Some state-specific codes that did not duplicate CPT were preserved as HCPCS Level II; all remaining state-specific codes were retired. As of 2023, fewer than 300 of 10,000+ HCPCS Level I and Level II codes describe interventions delivered by 2+ million non-allopathic providers authorized to manage patient care and bill without physician authorization. HCPCS I codes are primarily designed to describe physician-delivered or physician-managed care and do not reflect the breadth, clinical nuance, or legal distinctions of care delivered by millions of licensed non-allopathic providers. HCPCS II codes are primarily used to process claims for Medicare and Medicaid services and supplies not found in HCPCS I. If HCPCS I develops a code that is then duplicated by the AMA, the HCPCS II code is retired.

### Consequences of the Coding Gap

The absence of comprehensive coding for non-allopathic interventions produces systemic deficiencies including:
1. Inability to accurately bill and obtain reimbursement for non-allopathic services
2. Inability to compare clinical efficacy of non-allopathic care against conventional medical care
3. Absence of outcome-tracking infrastructure for non-allopathic interventions
4. Arbitrary and inconsistent payer reimbursement policy rather than standardized pricing
5. Inadequate fraud detection infrastructure for non-allopathic billing due to variations in scope of practice and practitioner type across over 50 legal boundaries (states, DC, and U.S. territories).

### Rising False Claims Act Liability in Healthcare Billing

The False Claims Act (FCA), 31 U.S.C. § § 3729-3733, imposes severe civil liability on providers and payers who knowingly submit—or cause the submission of—false or fraudulent claims to federal programs like Medicare, Medicaid, or TRICARE. Liability arises from knowingly false statements, reckless disregard for truth, or deliberate ignorance, with penalties including treble damages plus per-claim fines (currently over $25,000 adjusted for inflation). No specific intent to defraud is required—mere submission of unauthorized or inflated claims suffices if material to payment.

From fiscal years 2023-2025, FCA enforcement escalated dramatically: total settlements/judgments reached an all-time high of $6.8 billion in FY 2025 (ending Sep. 30, 2025), up from $2.9 billion in FY 2024, with healthcare accounting for over $5.7 billion (83% of total). Key fraud drivers include medically unnecessary services, overbilling, kickbacks, and unsupported diagnoses—often tied to billing outside legal scope or without proper authorization. Qui tam lawsuits surged to 1,297 in FY 2025, the highest ever, signaling heightened whistleblower scrutiny.

Non-allopathic providers face acute risk: recent cases illustrate liability for improper billing. For instance, in April 2026, Oasis Acupuncture LLC and its owner settled for $100,000 under the FCA for allegedly inflating claims to the VA for acupuncture services (e.g., reinsertion billing discrepancies), highlighting how alternative providers can trigger violations when claims misrepresent services or exceed scope. Broader trends show DOJ targeting unnecessary procedures, substandard care, and compliance misrepresentations—precisely where jurisdiction-specific Scope-of-Practice gaps expose providers to treble damages, exclusion from federal programs, and reputational harm.

The present invention mitigates these risks by embedding real-time Scope-of-Practice validation, AI-driven confidence scoring, and human review routing before EDI transmission. This prevents unauthorized interventions from reaching claims, reducing FCA exposure for non-allopathic providers (e.g., acupuncturists, naturopaths, midwives) who lack parity in coding infrastructure. By flagging ambiguous or pending regulatory changes and routing for review, the system addresses a core FCA vulnerability: implied certification of compliance with state/federal laws when submitting for reimbursement.

### Access to Care Barriers from Fragmented State Scope-of-Practice Laws

Despite the presence of over two million non-allopathic healthcare providers—such as nurse practitioners (over 461,000 licensed), physical therapists (around 635,000), chiropractors (about 66,000 businesses with many practitioners), acupuncturists (~34,000 licensed), naturopathic doctors (~6,000), certified nurse-midwives (~17,000), behavioral health providers (over 800,000 including licensed professional counselors, social workers, psychologists, and mental health counselors), massage therapists (approximately 321,000), somatic educators and therapists, functional and alternative medicine doctors (approximately 200,000), occupational therapists, pharmacists, and spiritual care providers—across the United States, patients face widespread barriers to timely, affordable care. The U.S. healthcare system grapples with ongoing access issues: as of December 2025 (latest HRSA data), there are 8,466 Primary Care Health Professional Shortage Areas (HPSAs) affecting 92 million people—approximately 27% of the population. Rural areas account for a significant portion of designations, with millions waiting months for basic care. We need thousands more practitioners just to clear these shortages, amid projected physician gaps of 70,000+ by 2038.

A primary driver is the patchwork of 50+ state-specific scope-of-practice laws, which vary dramatically in restrictions on non-physician/non-allopathic providers. In restrictive states, practitioners require physician supervision, collaborative agreements, or transition-to-practice hours (often 1,000-4,000), creating administrative burdens, liability risks, and disincentives for independent practice. This underutilizes a workforce comprising over 40% of clinical professionals, exacerbating shortages—especially in rural and underserved areas where non-allopathic providers are more likely to serve.

These legal silos block reimbursement parity, as claims for non-allopathic interventions often fail due to unauthorized coding or scope mismatches, deterring providers from accepting patients or participating in insurance networks. The result: reduced patient access, longer waits, higher costs, and inequitable outcomes, even as evidence shows expanded scope (e.g., full practice authority for NPs) lowers outpatient costs by 11-16% and maintains care quality without increased risk.

The present invention directly overcomes these barriers by automating jurisdiction-specific scope validation at the point of claim creation—ensuring only authorized interventions are billed, mapped, and transmitted via HIPAA-compliant EDI. This enables non-allopathic providers to deliver and document care compliantly, without payer-side rework or denial, thereby expanding access: patients gain more options from licensed practitioners, rural gaps shrink through better reimbursement flow, and the two-million-strong workforce finally achieves administrative parity with physicians. By preventing unauthorized claims and flagging regulatory ambiguities, the system also mitigates False Claims Act risks while promoting broader, safer utilization of holistic and alternative care.

### Scope-of-Practice Compliance Requirements

Federal and state regulations mandate that healthcare services be reimbursed only when delivered within a provider's legal scope of practice. State Scope-of-Practice regulations are complex, vary materially by jurisdiction, and are subject to frequent amendment through legislative and regulatory action. Without a mechanism for dynamically mapping billable intervention codes to current, jurisdiction-specific Scope-of-Practice law at the point of provider contracting, claim creation, during routing or before payment, insurers risk paying for care that is unauthorized under applicable law, while practitioners'risk legal liability for billing fraud and False Claims Act violations.

### The Rise of AI-Assisted Clinical Documentation

The increasing deployment of artificial intelligence tools in clinical settings—including speech recognition systems, ambient listening devices, clinical transcription services, and large language model (LLM)-based documentation workflows—has introduced new risks to claim integrity. These tools may generate, summarize, or pre-code encounter data without jurisdiction-specific validation tied to provider type, licensure and/or training. If AI validation is not integrated into claim workflows, AI-assisted claim preparation may cause widespread unauthorized procedures, incorrect coding, unsupported pricing, and non-compliant claims. AI-assisted claim preparation works for most physician claims due to harmonized scope of practice across state lines and physician insight into procedures but is very risky for coding non-allopathic care due to scope variations and lack of precision terminology to describe care.

### EDI Integration Gap

Efforts to modernize healthcare reimbursement have emphasized standardization through ANSI ASC X12N electronic data interchange (EDI) transaction formats, including the 837 (claim submission), 835 (remittance advice), and 276/277 (claim status inquiry/response) transaction sets. Non-allopathic services are largely excluded from these digital channels due to the absence of precision coding for 80+% of non-allopathic interventions.

### Prior Art and Its Limitations

U.S. Patent Application Publication No. US20150379241A1 discloses an automatic medical coding system employing natural language processing (NLP) to identify clinical information within narrative documentation and recommend CPT, ICD-10, or other standard codes for billing purposes. While this system improves coding speed and consistency for physician-generated documentation, it presents material limitations relevant to the present invention:
1. The system is optimized for physician-style narrative documentation and does not address the distinct documentation patterns, coding terminology requirements, or pricing needs of non-allopathic providers.
2. The system does not validate a licensed practitioner's legal authority to perform a billed procedure based on licensure type, training credentials, or geographic jurisdiction, creating substantial compliance risk under federal and state Scope-of-Practice mandates.
3. The system does not incorporate ABC codes, Relative Value Units (RVUs) for non-allopathic procedures, or mapping to Universal Product Codes (UPCs) for associated supplies and products—all of which are essential to accurate pricing and reimbursement of non-allopathic patient encounters.
4. The system does not include jurisdiction-specific, dynamically updated Scope-of-Practice logic capable of validating claim content against current state law before EDI transmission.

### Need for the Present Invention

Accordingly, a substantial and unmet need exists for a system and method that:
1. Encodes non-allopathic interventions with clinical, legal, and economic specificity using a recognized and comprehensive code set
2. Validates encoded interventions against current, jurisdiction-specific provider Scope-of-Practice law
3. Assigns accurate pricing using standardized mechanisms (RVUs for procedures; UPCs for products)
4. Integrates an AI-assisted intake and normalization layer with legally aware validation
5. Maps validated codes to HIPAA-mandated code sets for compliant EDI transmission
6. Routes uncertain or non-compliant outputs for human review before claim creation

The present invention addresses each of these needs.

### SUMMARY OF THE INVENTION

The present invention provides a computerized method and system for encoding, validating, pricing, and electronically processing healthcare interventions delivered by non-allopathic healthcare providers. The system integrates ABC codes (a comprehensive coding system specifically developed to describe non-allopathic interventions) with jurisdiction-specific Scope-of-Practice validation, standardized pricing mechanisms, and an artificial intelligence intake and normalization layer, enabling compliant electronic claim submission through HIPAA-mandated EDI transaction sets, if available. In certain embodiments, the system further includes a legal classification layer that maps standardized procedure codes to legally defined intervention categories, enabling Scope-of-Practice validation at a classification level rather than at an individual code level. In light of the 2026 HIPAA final rule adopting X12 Version 6020 for claims attachments (effective May 26, 2026; mandatory compliance May 26, 2028), the system supports the 275 Claim Attachment transaction for both solicited and unsolicited attachments using LOINC codes to identify document types (no registration required for LOINC use).

In a first aspect, the invention provides a computer-implemented system comprising: an ABC Code Database storing codes for non-allopathic healthcare interventions; a Scope-of-Practice Validation Engine configured to determine provider authorization for encoded interventions based on provider type, licensure, and geographic jurisdiction; a pricing engine assigning RBRVS or RVU values to procedures and UPCs to products; a Code Mapping Module translating validated ABC code to billable code; an AI intake and normalization layer processing structured and unstructured encounter data, if available; and an EDI transaction generator producing compliant ANSI ASC X12N 837 transactions.

In a second aspect, the invention provides a computer-implemented method comprising: receiving encounter data; extracting candidate interventions; recommending billable codes; validating provider authority against jurisdiction-specific Scope-of-Practice rules; assigning pricing; mapping to HCPCS/CPT codes when needed; generating compliant EDI transactions; and routing low-confidence outputs for human review.

### BRIEF DESCRIPTION OF THE DRAWINGS

FIG. 1 is a high-level system architecture diagram illustrating the principal components of the non-allopathic healthcare transaction processing system, including the Input Sources 101, AI intake layer 110, ABC Code Database 120, Scope-of-Practice Validation Engine 130, Pricing Engine 140, Code Mapping Module 150, EDI Transaction Generator 160, and Payer and/or Clearinghouse System.

FIG. 2 is a flowchart illustrating the method steps of the AI Intake and Normalization 110 process, including receipt of raw encounter data; structured records, free-text clinical notes, audio transcriptions, SOAP notes, and payer edits; extraction of valid candidate interventions; and routing logic for low confidence interventions to Human Review Interface 270.

FIG. 3 is a diagram illustrating the Scope-of-Practice Validation Engine 130, showing the relationship between provider type database, jurisdiction-specific regulatory database, intervention database, and validation output.

FIG. 4 is a diagram illustrating the Pricing Engine 140, showing assignment of Resource-based Relative Value Scale (RBRVS) total RVU or other relative value systems such as Relative Values for Physicians RVU for procedure pricing and Universal Product Codes (UPCs) for product and supply pricing.

FIG. 5 is a block diagram illustrating the integration of the system with HIPAA ANSI ASC X12N EDI transaction sets, including but not limited to the 837P/837I and/or 275 Claim Attachment submission transactions, 275 Claim Attachment, 835 remittance advice transaction, 276/277 claim status transaction, and the Audit/Log Database.

FIG. 6 is a flowchart illustrating the complete AI claim creation workflow from Input Sources 101 to EDI transmission, including decision nodes for high/low confidence scoring and Human Review Interface 270 routing.

FIG. 6A is a continuation of the complete AI claim creation workflow from FIG. 6. And illustrates requirements for EDI Generation Transaction 160, Human Review Interface 270, Payer and/or Clearinghouse System 170, Payer Responses, and event logging for audit database.

FIG. 7 is a structural diagram illustrating the ABC Code Database 120, showing the data fields stored for each ABC code record including code identifier, code description, provider type, jurisdiction flags, pricing type, HCPCS mapping, match type, version, and last updated timestamp. FIG. 7 further illustrates the three downstream routing paths from the ABC Code Database: procedure codes 730 routed to the Scope-of-Practice Validation Engine 130 and RVU pricing module 141; product codes 730 routed to the UPC cross-reference module 142 and pricing database; and unmapped codes routed to the unmapped code holding queue 740 for human review, supplemental documentation generation and possible code maintenance.

FIG. 8 is a process diagram illustrating the jurisdiction-specific regulatory database update cycle 800, showing the flow of regulatory information from external sources including state board regulations 811, Statutes/Administrative Regulations/Case Law 812, US Licensing 813, and CMS Policy 814, through the regulatory monitoring module 820 and change detection and classification module 830, to the jurisdiction-specific regulatory database 132, including branching logic distinguishing confirmed regulatory changes from pending or contested changes, and a provider notification output 840.

FIG. 9 is a workflow diagram illustrating the human review interface 270, showing the three entry points into the review queue 910 from confidence score low AI score 260, Scope-of-Practice review-required flags 137, and flag missing data elements 245; the reviewer workstation display 920; the three reviewer decision paths of new code input authorized 930, missing data elements 935, and reject 940; the approve log decision module 960; rejection log decision module 970, and rejection reason notification sent 980.

### DETAILED DESCRIPTION OF THE INVENTION

### Overview

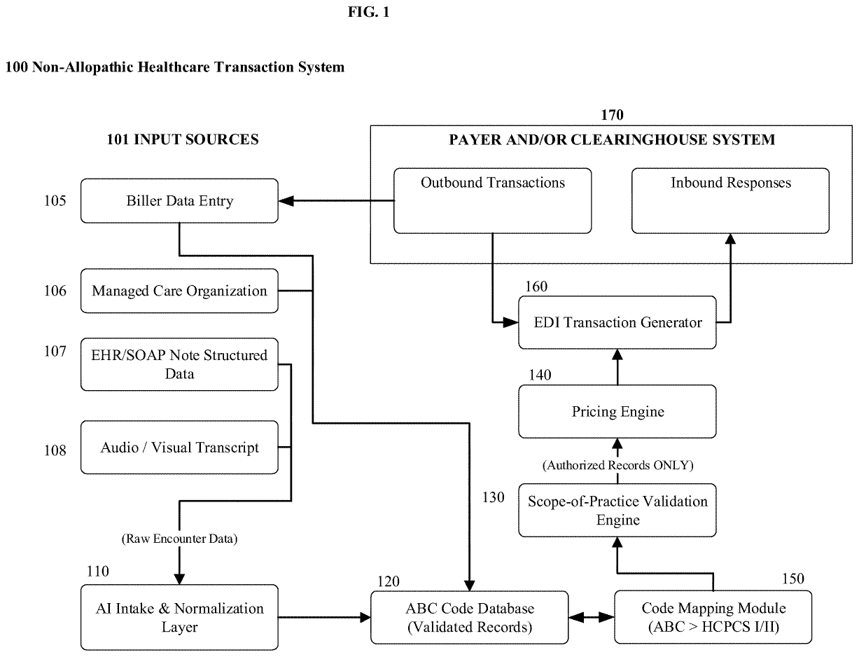

Referring now to the drawings, wherein like reference numerals designate corresponding parts throughout, FIG. 1 illustrates the overall system architecture of the non-allopathic healthcare transaction processing system 100. System 100 comprises Input Sources 101, an AI intake and normalization layer 110, an ABC Code Database 120, a Scope-of-Practice Validation Engine 130, a Pricing Engine 140, a Code Mapping Module 150, an EDI transaction generator 160, and Payer System 170. The system may further include a legal classification layer configured to group standardized procedure codes into legally defined intervention categories for use in Scope-of-Practice validation.

### AI Intake and Normalization Layer (110)

Referring to FIGS. 1, 2, and 3, the AI intake and normalization layer 110 is configured to receive encounter data from a plurality of input sources including, without limitation: structured electronic health record (EHR) data, free-text clinical notes, audio transcriptions from speech recognition systems, ambient listening platforms, clinical transcription services, SOAP (Subjective, Objective, Assessment, Plan) notes, large language model (LLM)-generated documentation, payer edit responses, and prior authorization records.

The AI intake layer 110 employs natural language processing (NLP) and machine learning models to:
1. Parse and normalize received encounter data regardless of input format or source
2. Extract candidate healthcare interventions, procedures, and products from normalized data
3. Identify the provider type and licensure information associated with the encounter
4. Identify the geographic jurisdiction applicable to the encounter
5. Recommend one or more billing codes (currently ABC and HCPCS I and II) corresponding to extracted candidate interventions and diagnostic code(s) and/or DRGs
6. Assign a confidence score to each recommended ABC code
7. Identify missing data elements required for complete claim creation
8. Route records with confidence scores below a configurable threshold to the human review interface 270 before claim creation proceeds.

The AI Intake layer 110 is specifically distinguished from prior art automated coding systems by its integration with the Scope-of-Practice Validation Engine 130, which prevents AI-recommended codes from propagating into claim transactions without jurisdiction-specific legal validation.

### ABC Code Database (120)

The ABC Code Database 120 stores a comprehensive, structured repository of all alphabetic ABC codes (originally known as Alternative Billing Concepts). ABC codes are a coding system originally developed and first published in 1998, originally designed to describe interventions, products, and services delivered by alternative healthcare providers and then expanded in 1999 to describe nursing, behavioral health, chaplaincy, spiritual care nursing and indigenous medicine. ABC Code Database 120 includes, for each stored code:
1. A unique alphanumeric ABC code identifier
2. A 5-character all-alphabetic ABC code
3. A short description of the procedure or supply item
4. A clinical description of the associated procedure, product, or service
5. Provider type associations indicating which non-allopathic provider types are licensed or trained to perform the coded intervention
6. Applicable geographic jurisdiction flags
7. Associated RVU values (for procedures) or UPC cross-references (for products)
8. Mapping relationships to corresponding HCPCS Level I (CPT) or HCPCS Level II codes where applicable
9. Version control and update timestamps.

The ABC Code Database 120 is maintained with regular updates to reflect additions, revisions, moves within the hierarchy and retirements of codes, and is structured to support efficient query and retrieval by the AI Intake layer 110, the Scope-of-Practice Validation Engine 130, and the Code Mapping Module 150.

### Scope-of-Practice Validation Engine (130)

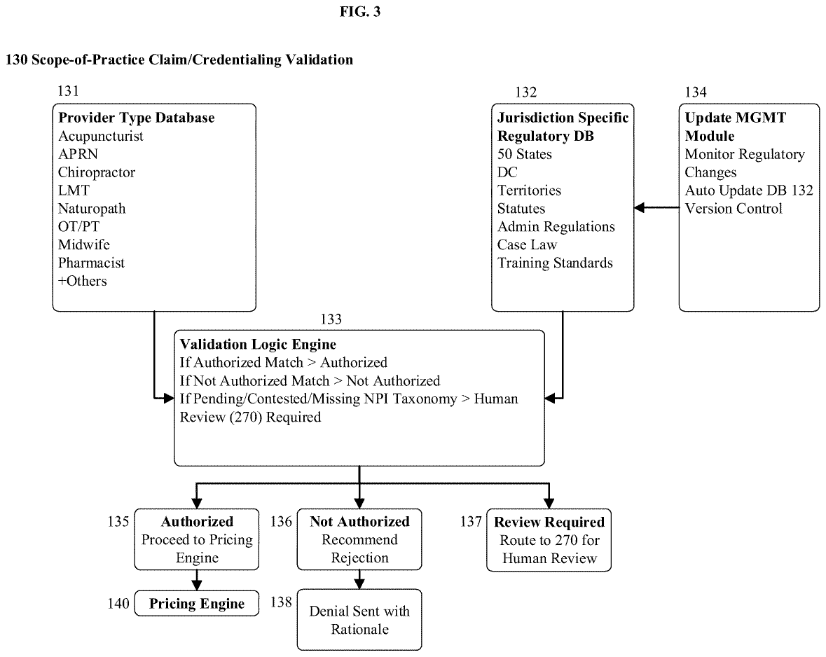

Referring to FIG. 3, the Scope-of-Practice Validation Engine 130 is a critical component of the present invention that addresses a fundamental deficiency in prior art systems. The Scope-of-Practice Validation Engine 130 comprises:
1. A provider type database 131 storing classifications of non-allopathic provider types (e.g., acupuncturist, advanced practice registered nurse, chiropractor, licensed massage therapist, licensed midwife, naturopathic doctor, occupational therapist, physical therapist).
2. A jurisdiction-specific regulatory database 132 storing current scope-of-practice rules, statutes, administrative regulations, case law, training standards and administrative guidance for each non-allopathic provider type in each of the fifty (50) U.S. states, the District of Columbia, and U.S. territories.
3. A validation logic engine 133 that, for a given combination of: provider type, geographic jurisdiction, and proposed ABC-coded intervention; determines whether the provider is legally authorized to perform the intervention under applicable current law.
4. An update management module 134 that monitors regulatory changes and updates the jurisdiction-specific regulatory database 132 to reflect current law.

The Scope-of-Practice Validation Engine 130 receives as input:
a. the provider type from the provider type database 131;
b. the geographic jurisdiction;
c. one or more ABC-coded candidate interventions recommended by the AI intake layer 110. The validation logic engine 133 queries the jurisdiction-specific regulatory database 132 and returns one of three validation decisions for each candidate intervention:
5. AUTHORIZED 135: The provider type is legally authorized to perform the intervention in the applicable jurisdiction proceeds to Pricing Engine 140.
6. NOT AUTHORIZED 136: The provider type is not legally authorized to perform the intervention in the applicable jurisdiction; the intervention is flagged and recommended for rejection with Denial Rationale sent back to requester 138.
7. REVIEW REQUIRED 137: The authorization status is ambiguous, contested, not supported by NPI taxonomy code or subject to pending regulatory change; the intervention is routed to the human review interface 270.

### Legal Classification Layer for Standardized Code Sets

In certain embodiments, the system includes a legal classification layer configured to associate one or more standardized procedure codes with a higher-level legal intervention category. For example, a plurality of standardized procedure codes, including but not limited to CPT or HCPCS codes describing minor surgical procedures, may be mapped to a single internal ABC code representing a legally defined intervention category (e.g., “minor surgery”).

This internal classification is stored within the Code Maintenance Database (CMDB) and is not required to be published as part of an external coding manual.

The Scope-of-Practice Validation Engine evaluates provider authorization at the level of the internal classification rather than at the level of individual standardized codes. This enables the system to: apply consistent legal validation across multiple related procedure codes; determine whether a provider type is authorized to perform a class of interventions in billable codes; allow or restrict use of standardized procedure codes based on legal authorization associated with the classification; and support use of standardized codes by non-allopathic providers when legally permissible under jurisdiction-specific rules.

In this manner, the system provides a legal abstraction layer that governs the permissible use of standardized healthcare procedure codes.

### Pricing Engine (140)

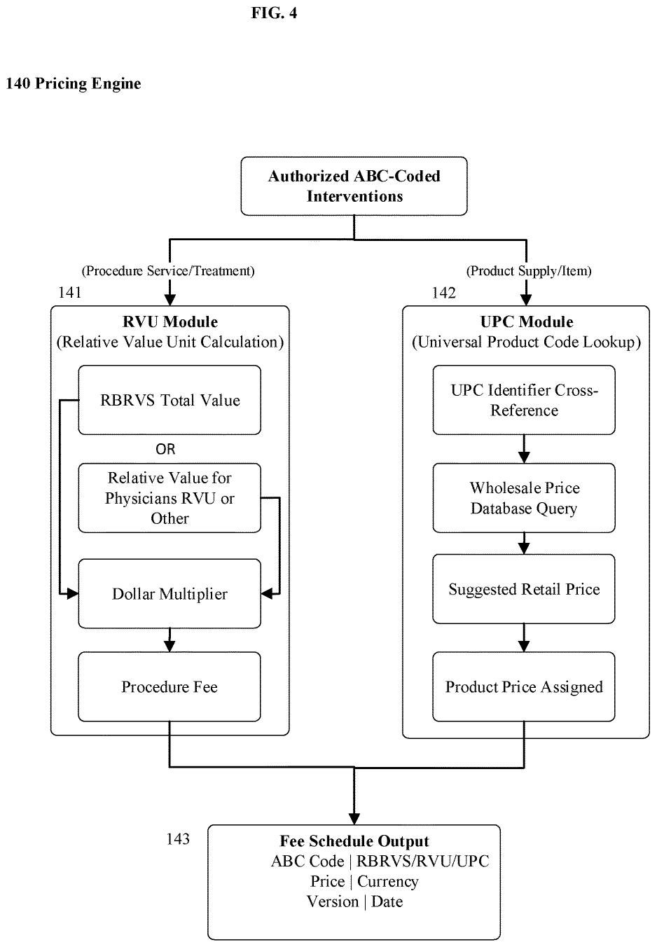

Referring to FIG. 4, the Pricing Engine 140 assigns standardized pricing to validated, ABC-coded interventions and associated products. The Pricing Engine 140 operates through two distinct pricing mechanisms:

RVU-Based Procedure Pricing: For ABC-coded procedures and interventions, the Pricing Engine 140 assigns Relative Value Units (RVUs) using Resource-Based Relative Value Scale (RBRVS) methodology or Relative Value for Physician methodology. RBRVS assignments for non-allopathic procedures incorporate work RVUs reflecting the clinical effort, skill, and time required; practice expense RVUs; and malpractice RVUs. RVU-based pricing enables legally defensible fee schedules for non-allopathic procedures and supports cross-profession cost comparisons by payers and health systems. If the billable intervention is a HCPCS code, then the HCPCS RBRVS value is assigned.

UPC-Based Product Pricing: For ABC-coded products, supplies, and prescribed nutraceuticals, the Pricing Engine 140 cross-references Universal Product Codes (UPCs) to current wholesale and suggested retail pricing databases, enabling accurate and verifiable pricing for physical products dispensed or prescribed by non-allopathic providers used in patient treatment.

### Code Mapping Module (150)

The Code Mapping Module 150 translates validated, priced ABC codes into the HCPCS Level I (CPT) or HCPCS Level II codes currently mandated under HIPAA Transaction and Code Set standards for outpatient electronic claim submission. This mapping step ensures that non-allopathic interventions encoded with clinical and legal specificity using ABC codes and/or HCPCS codes can be transmitted through existing HIPAA-compliant EDI infrastructure without requiring payers or trading partners to independently implement ABC code processing capability. This module lets the practitioner select an ABC code for documentation and pricing and then maps the ABC code to the closest possible HCPCS I or II code for billing. If the ABC code is more granular than the HCPCS code, then the ABC code and description are used to supplement the HCPCS code.

The Code Mapping Module 150 maintains a regularly updated crosswalk database linking ABC codes to their closest corresponding HCPCS codes, including notation of:
1. Direct equivalents (one-to-one mapping)-use HCPCS code, only, to document care
2. Partial equivalents with specificity differences (flagged for supplemental Documentation and pricing)
3. ABC-coded interventions with no current HCPCS equivalent (flagged for supplemental documentation and pricing and mapped to unlisted or not otherwise specified HCPCS code).

In embodiments where ABC codes are formally integrated into U.S. EDI standards, the mapping step to HCPCS for partial or no equivalent HCPCS performed by the Code Mapping Module 150 will be rendered operationally redundant, and the system is then configured to transmit ABC codes directly into the same EDI transactions as HCPCS codes.

### EDI Transaction Generator (160)

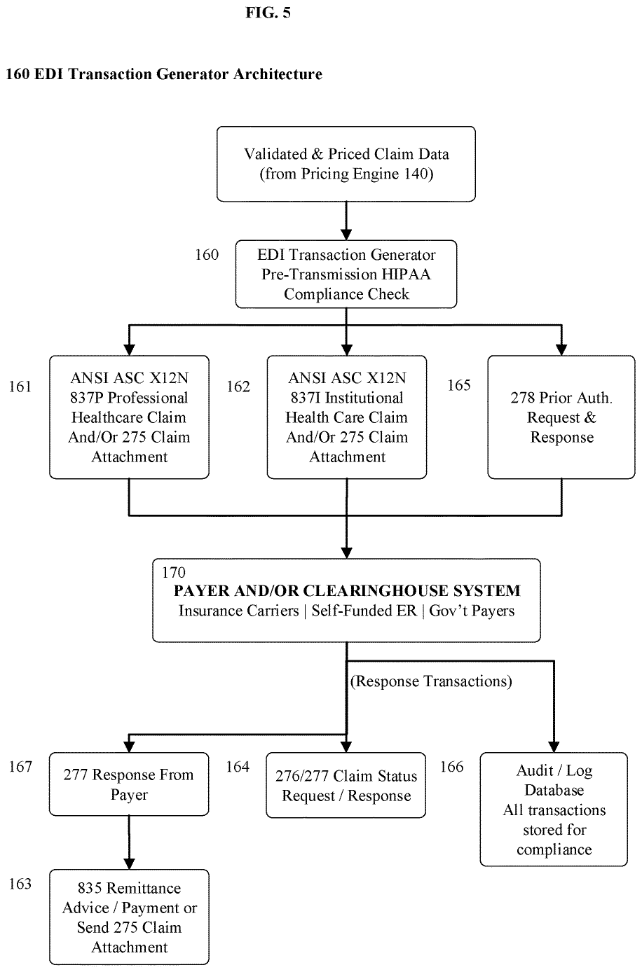

Referring to FIG. 5, the EDI Transaction Generator 160 produces complete, compliant ANSI ASC X12N EDI transactions incorporating validated, priced, and mapped claim data. The EDI Transaction Generator 160 supports:
1. 837P/837I: Professional and Institutional Health Care Claim transactions
2. 835: Health Care Claim Payment/Advice transactions
3. 275 Claim Attachments
   a. The 2026 HIPAA final rule adopting X12 Version 6020 for claims attachments (effective May 26, 2026; mandatory compliance May 26, 2028), the EDI transaction generator supports the 275 Claim Attachment transaction for both solicited (payer-requested via 277) and unsolicited attachments. Attachment content may include structured HL7 C-CDA documents or unstructured files (e.g., PDFs), with LOINC codes identifying the document type—no registration required for LOINC use. For non-standard interventions like ABC codes, the system embeds supplemental details (clinical descriptions, pricing rationale) within the 275 Claim Attachment, linked to the originating claim via trace numbers, while mapping core billables to HCPCS for compliant 837 transmission.
4. 276/277: Health Care Claim Status Request and Response transactions
5. 278: Health Care Services Review—Request for Review and Response (prior authorization).

The EDI Transaction Generator 160 populates all required data elements of each transaction type from the validated claim data produced by preceding system components and performs final pre-transmission compliance checking against applicable HIPAA Implementation Guide requirements.

### Complete AI Claim Workflow (600)

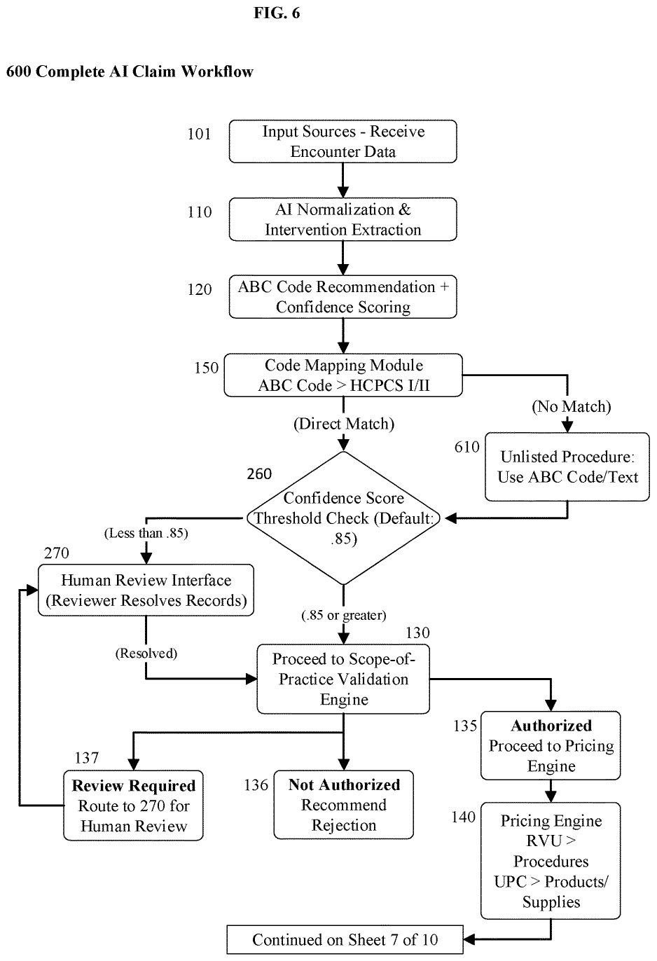

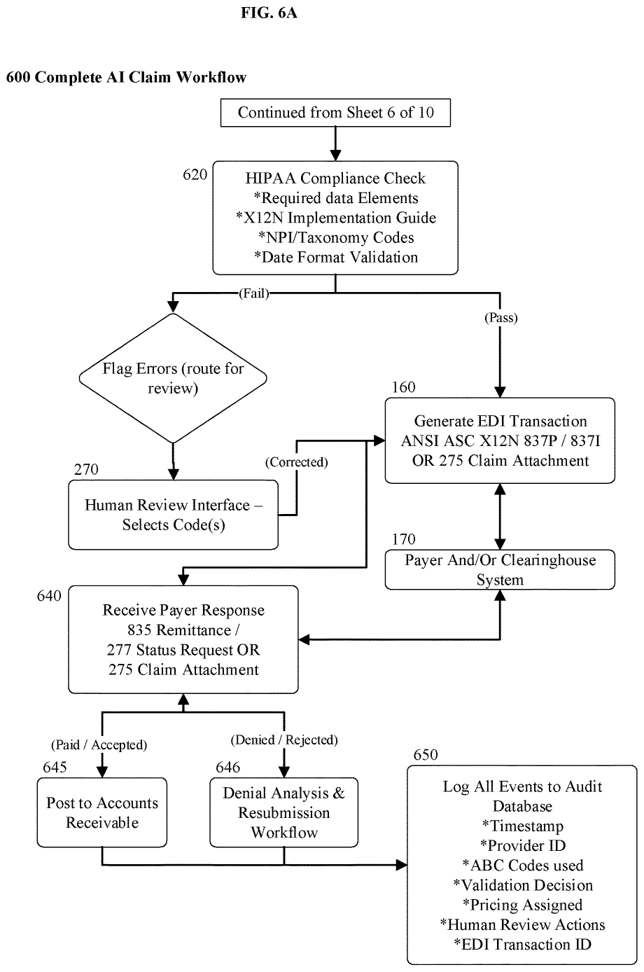

Referring to FIG. 6, the complete claim creation workflow proceeds as follows:
1. Encounter data from Input Sources 101 is received by the AI intake and normalization layer 110 from one or more input sources
2. The AI intake layer 110 normalizes the data, extracts candidate interventions, and recommends ABC codes with confidence scores
3. The Code Mapping Module 150 maps validated, priced ABC codes to HCPCS codes for EDI transmission. Matches move to Confidence Score Decision 260 and No Match are directed to Unlisted Procedure codes 610
4. Low-confidence recommendations are routed to the human review interface 270; High-confidence recommendations proceed to validation
5. The Scope-of-Practice Validation Engine 130 validates each recommended ABC code against provider type, licensure, and geographic jurisdiction
6. AUTHORIZED 135 interventions proceed to pricing; NOT AUTHORIZED 136 interventions are removed; REVIEW REQUIRED 137 interventions are routed to human review interface 270
7. The Pricing Engine 140 assigns RVU-based pricing to authorized procedures and UPC-based pricing to authorized products.

### Human Review Interface (270)

The Human Review Interface 270 on FIG. 6, provides a structured workflow for clinical coding specialists, billing staff and managed care compliance reviewers to evaluate and resolve claim records routed for human review by the AI intake layer 110 or the Scope-of-Practice Validation Engine 130. Records may be routed for human review based on low AI confidence scores, ambiguous Scope-of-Practice authorization status, missing required data elements (such as taxonomy codes within a practitioner NPI, or unusual pricing variances. The Human Review Interface 270 logs all reviewer decisions and supporting rationale for audit purposes.

Referring to FIG. 6A, the continued claim creation workflow proceeds as follows:
1. HIPAA Compliance Check 620 performed to meet requirements; Failure routes to Human Review 270; Pass routes to EDI Transaction 160
2. The EDI Transaction Generator 160 assembles and transmits compliant ANSI ASC X12N 837 transactions, and/or 275 Claim Attachments
3. All workflow events are logged for audit 650, compliance reporting, and outcomes analysis.

### ABC Code Database Structure (700)

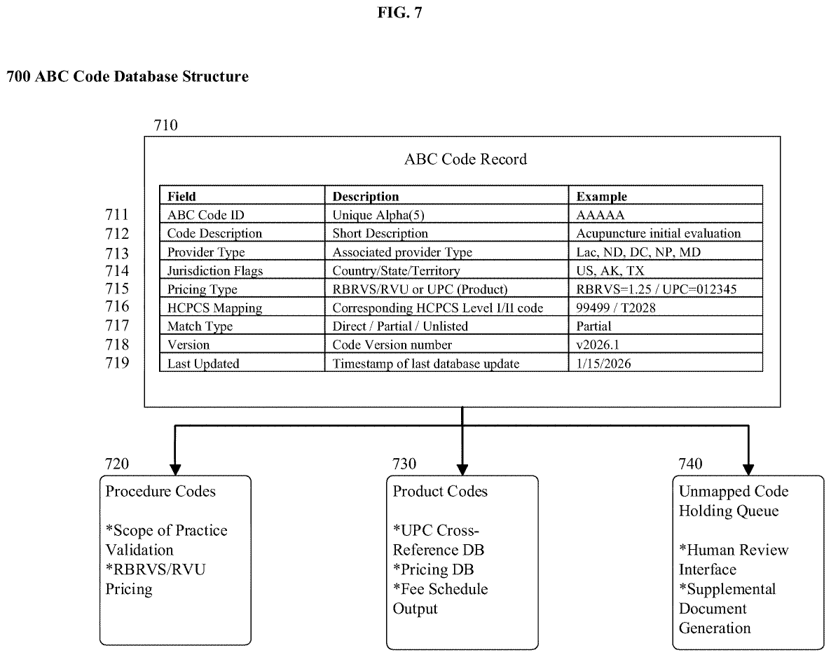

Referring now to FIG. 7, the ABC Code Database 120 is described in structural detail. The ABC Code Database 120 comprises a relational database management system storing a plurality of ABC code records 710. Each ABC code record 710 contains the following discrete data fields:

Field 711—ABC Code Identifier: A unique alphanumeric identifier assigned to each distinct non-allopathic healthcare intervention, product, or service encoded in the system. The identifier follows a structured format enabling programmatic sorting and retrieval by provider type category, intervention category, and jurisdiction applicability.

Add a field for five-character ABC code.

Field 712—Code Description: A standardized textual description of the healthcare intervention, product, or service associated with the ABC code. Clinical descriptions are authored to reflect current clinical terminology used by non-allopathic providers in practice documentation and are updated periodically to reflect evolving clinical standards and terminology.

Field 713—Provider Type: One or more designations identifying the non-allopathic provider type or types recognized as performing the coded intervention. Provider type designations include but are not limited to: Licensed Acupuncturist (LAc), Doctor of Chiropractic (DC), Licensed Massage Therapist (LMT), Certified Nurse-Midwife (CNM), Naturopathic Doctor (ND), Occupational Therapist (OT), Physical Therapist (PT), Licensed Professional Counselor (LPC), Registered Dietician (RD), Clinical Pharmacist, and Spiritual Care Provider. The provider type association field is used as a primary input to the Scope-of-Practice Validation Engine 130.

Field 714—Jurisdiction Flags: One or more geographic jurisdiction designations indicating the U.S. states, territories, or federal programs in which the coded intervention is recognized for billing purposes. A jurisdiction flag value of ALL indicates the intervention is universally recognized across all domestic jurisdictions. State-specific flags restrict applicability to named jurisdictions and trigger jurisdiction-specific Scope-of-Practice validation queries.

Field 715—Pricing Type: A designation of either RVU (Relative Value Unit) for procedure and service codes or UPC (Universal Product Code) for product and supply codes, directing the Pricing Engine 140 to apply the appropriate pricing methodology. For RVU-designated codes, this field stores the assigned RVU value. For UPC-designated codes, this field stores the UPC cross-reference identifier.

Field 716—HCPCS Mapping: The corresponding HCPCS Level I (CPT) or HCPCS Level II code to which the ABC code is mapped for EDI transmission purposes. This field is populated where a mapping relationship exists and is left null where no current HCPCS equivalent exists, triggering routing to the unmapped code holding queue 840.

Field 717—Match Type: A classification of the quality of the HCPCS mapping relationship, designated as one of: DIRECT (one-to-one clinical equivalence), PARTIAL (partial clinical equivalence with noted specificity differences), or NO MATCH (no current HCPCS equivalent exists). PARTIAL and NO MATCH designations trigger supplemental documentation generation or unlisted procedure code selection before routing.

Field 718—Version: The version identifier of the ABC code record, enabling tracking of code additions, revisions, and retirements across database update cycles.

Field 719—Last Updated: A timestamp recording the date and time of the most recent update to the ABC code record, enabling audit trail maintenance and regulatory compliance documentation.

As illustrated in FIG. 7, the ABC Code Database 120 routes records to three downstream processing paths based on code type and mapping status:
1. Procedure Codes 720: Codes designated as RVU-type procedures are routed to the Scope-of-Practice Validation Engine 130 for provider authorization confirmation, and subsequently to the RVU pricing module 141 for fee assignment.
2. Product Codes 730: Codes designated as UPC-type products are routed to the UPC cross-reference module 142 for product identification and current pricing database query, and subsequently to the fee schedule output 143.
3. Unmapped Code Holding Queue 740: Codes carrying a NO MATCH designation in field 717 are routed to the unmapped code holding queue 740, from which they are presented to the human review interface 270 for manual coding resolution, unlisted procedure code assignment, or supplemental documentation generation before claim creation proceeds.

### Jurisdiction-Specific Regulatory Database Update Cycle (800)

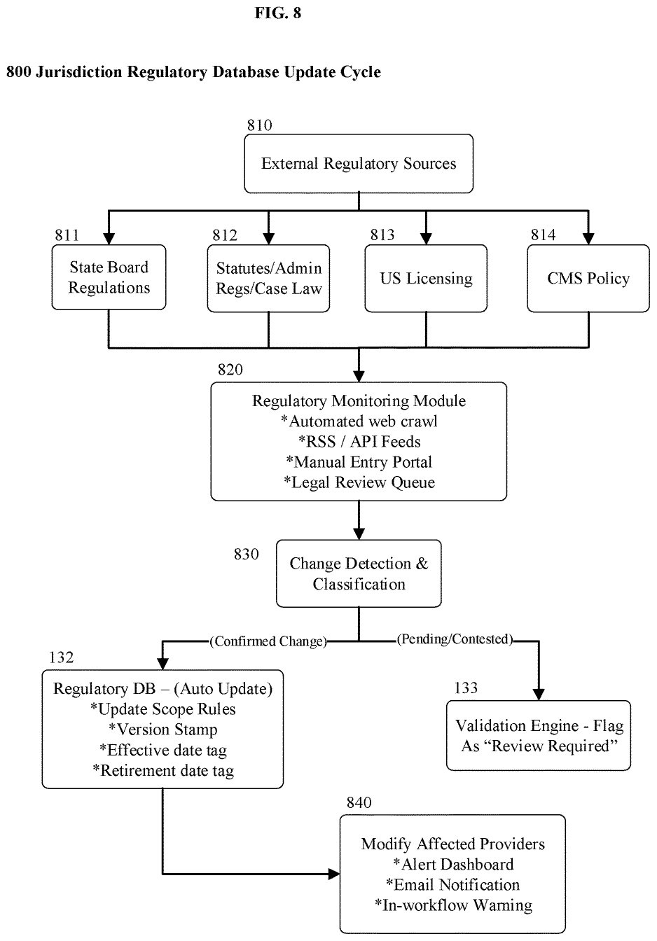

Referring now to FIG. 8, the jurisdiction-specific regulatory database update cycle 800 ensures that the Scope-of-Practice Validation Engine 130 operates against current, accurate, and legally defensible Scope-of-Practice rules at all times. The update cycle 800 is a continuous automated process supplemented by manual legal review, comprising the following components:
1. External Regulatory Sources 810: The update cycle 800 monitors four categories of external regulatory sources:
2. State Board Regulations 811: Administrative rules and regulations published by state professional licensing boards governing the scope of practice of each non-allopathic provider type in each jurisdiction. These sources are monitored through automated web crawling of official state board websites and regulatory publication portals.
3. Legislative Acts and Bills 812: State and federal legislation affecting non-allopathic provider scope of practice, including enacted statutes, pending bills, and enacted amendments to existing law. Legislative monitoring includes tracking bill status through introduction, committee review, floor vote, executive action, and enactment stages.
4. US Licensing 813: Centers for Medicare and Medicaid Services (CMS) program transmittals, manual updates, and policy guidelines affecting reimbursement eligibility and Scope-of-Practice recognition for non-allopathic providers under federal healthcare programs.
5. CMS Policy 814: Credentialing standards, Scope-of-Practice statements, and clinical guidelines published by national professional associations for non-allopathic provider types, which inform but do not independently govern the legal scope of practice.

Regulatory Monitoring Module 820: The regulatory monitoring module 820 aggregates information from all external regulatory sources 810 through a combination of automated monitoring mechanisms including web crawling, RSS feed subscriptions, regulatory API integrations where available, and a manual entry portal enabling legal and compliance staff to input regulatory changes identified through direct monitoring. All incoming regulatory information has timestamp, source-tagged, and queued for change detection processing.

Change Detection and Classification Module 830: The change detection and classification module 830 analyzes incoming regulatory information to identify, characterize, and classify changes relevant to non-allopathic provider Scope-of-Practice rules. Each identified change is classified as one of:
1. New Law: A newly enacted statute or regulation establishing or modifying Scope-of-Practice authorization for a provider type in a jurisdiction.
2. Amendment: A modification to an existing statute or regulation affecting existing Scope-of-Practice rules.
3. Repeal: A retirement or repeal of an existing statute or regulation affecting existing Scope-of-Practice rules.
4. Pending: A proposed change that has not yet been enacted, including pending legislation or proposed rulemaking.
5. Contested: A change whose legal effect is uncertain due to litigation, conflicting interpretations, or pending administrative proceedings.

Confirmed Changes: Regulatory changes classified as New Law, Amendment, or Repeal with confirmed effective dates are automatically propagated to the jurisdiction-specific regulatory database 132 by the update management module 134 described in FIG. 3. Each update is version-stamped with an effective date and, where applicable, a retirement date, enabling the validation logic engine 133 to apply historically accurate Scope-of-Practice rules to encounters dated prior to a regulatory change.

Pending and Contested Changes: Regulatory changes classified as Pending or Contested are not automatically propagated to the jurisdiction-specific regulatory database 132. Instead, these changes cause the validation logic engine 133 to flag affected provider type and intervention combinations with a REVIEW REQUIRED validation state 137, routing associated claim records to the human review interface 270 until the regulatory change is resolved and confirmed.

Provider Notification Module 840: Upon confirmation and database propagation of a regulatory change, the provider notification module 840 generates alerts to affected providers through the system dashboard, email notification, and in-workflow warning displays, advising of changes to their authorized scope of practice that may affect claim creation and reimbursement eligibility.

### Human Review Interface Workflow (900)

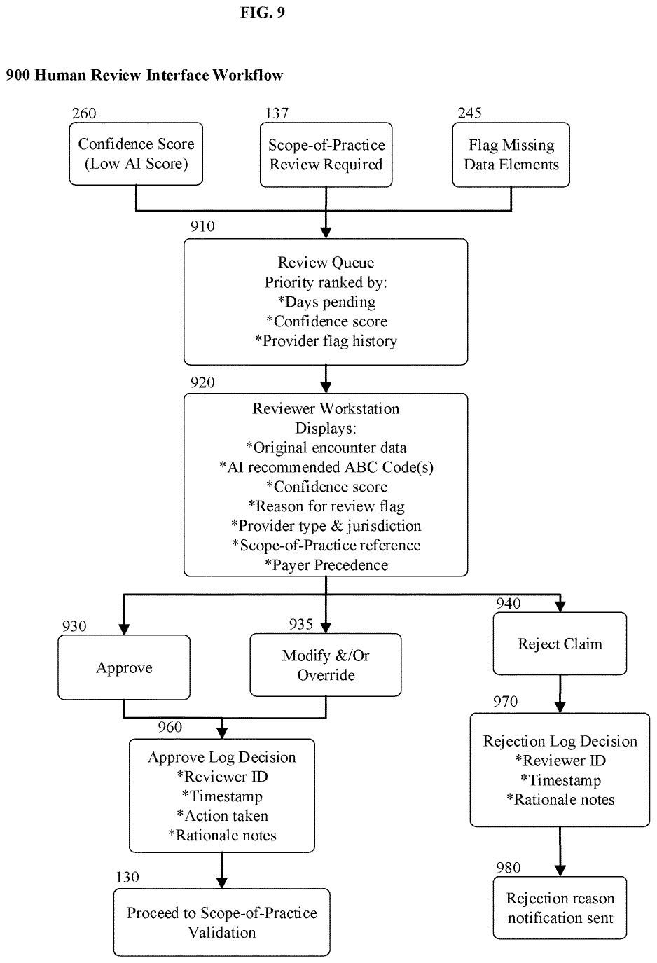

Referring now to FIG. 9, the human review interface 270 is described in operational detail. The Human Review Interface 270 serves as the critical quality control and compliance safeguard within the claim creation workflow, ensuring that no claim record with unresolved uncertainty, unauthorized coding, or missing data elements is transmitted as an EDI transaction without human review and resolution.

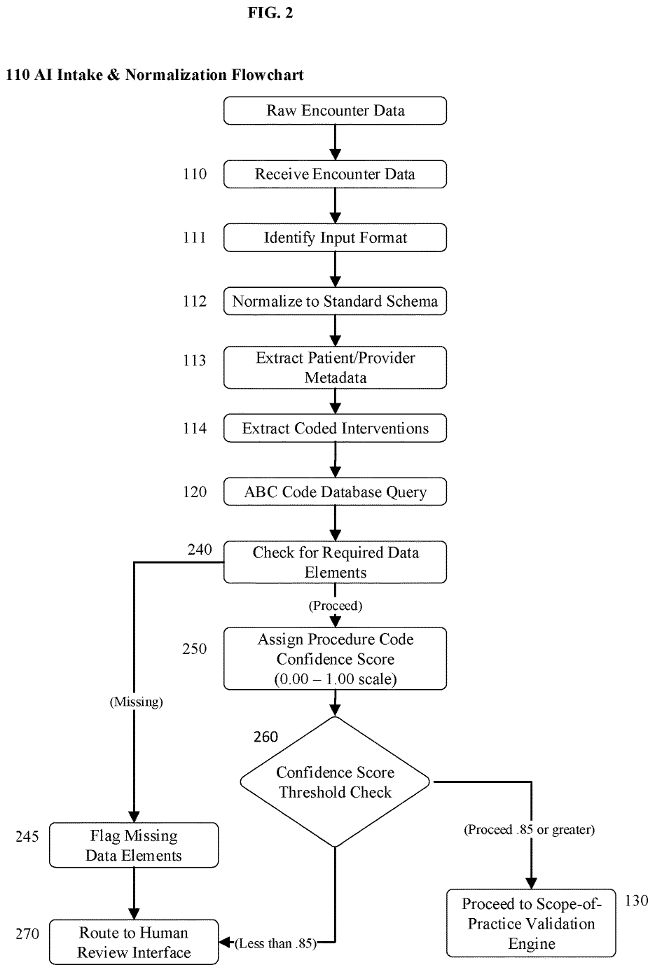

Review Queue 910: Records are routed to review queue 910 from three entry points within the claim creation workflow:
1. Low Confidence AI Score: Records for which the AI intake and normalization layer 110 assigns a confidence score below the configurable threshold (default: 0.85) as described in FIG. 2.
2. Scope-of-Practice REVIEW REQUIRED Flag: Records for which the Scope-of-Practice Validation Engine 130 returns a REVIEW REQUIRED validation state 137 as described in FIG. 3, including records affected by pending or contested regulatory changes.
3. Missing Data Elements Flag: Records for which required claim data elements are identified as absent or incomplete by the AI intake layer 110 as described at step 245 of FIG. 2.

Records in review queue 910 are prioritized for reviewer assignment based on a multi-factor ranking algorithm incorporating: claim monetary value, number of days the record has been pending review, AI confidence score, and the provider's historical flag rate. Higher-value, longer-pending, lower-confidence records from providers with elevated historical flag rates are assigned higher review priority.

Reviewer Workstation Display 920: The reviewer workstation presents clinical coding specialists and compliance reviewers with a consolidated display of all information relevant to resolving the flagged record, including:
1. The complete original encounter data in its source format
2. The AI-recommended ABC code or codes with associated confidence scores
3. The specific reason or reasons the record was flagged for review
4. The provider type, license number, and geographic jurisdiction
5. The applicable Scope-of-Practice reference materials from the jurisdiction-specific regulatory database 132
6. A curated list of similar previously resolved cases for reference
7. The HCPCS mapping relationship for the recommended ABC code or codes
8. Any pricing anomalies or variances identified by the Pricing Engine 140.

Reviewer Decision Paths: Following review of the displayed information, the reviewer selects one of three resolution actions:
1. Approve 930: The reviewer confirms the AI-recommended ABC code selection as accurate, complete, and compliant. The record proceeds to Approval Log Decision 960, then to the main claim workflow at the Scope-of-Practice Validation Engine 130.
2. Modify &/or Override 935: The reviewer overrides the AI-recommended ABC code selection and substitutes an alternative ABC code, corrects missing data elements, or resolves Scope-of-Practice ambiguity through documented legal interpretation. The modified record proceeds to Approval Log Decision 960, then to the main claim workflow at the Scope-of-Practice Validation Engine 130.
3. Reject Claim 940: The reviewer determines that the record cannot be resolved into a compliant, authorized claim and removes it from the claim creation workflow. Rejected records are logged into Rejection Log Decision 970, flagged for provider notification and returned to the provider for additional documentation or clinical clarification.

Approve Log Decision Module 960: All approve and modify decisions are recorded in the decision logging module 960 with the following data elements: reviewer identifier, timestamp, action taken, ABC code confirmed or substituted, and free-text rationale notes entered by the reviewer. Decision logs are maintained in the audit database and are available for compliance reporting, payer audit response, fraud investigation, and outcomes analysis.

Rejection Log Decision Module 970: All rejection decisions are recorded in the rejection log decision module 970 with the following data elements: reviewer identifier, timestamp, and free-text rationale notes entered by the reviewer. Decision logs are maintained in the audit database and are available for compliance reporting, payer audit response, fraud investigation, and outcomes analysis.

Rejection Reason 980 sends notification to applicable party.

### System Integration and Interoperability

The non-allopathic healthcare transaction processing system 100 is designed for interoperability with existing healthcare information technology infrastructure. In preferred embodiments, the system 100 is implemented as a cloud-based software-as-a-service (SaaS) platform accessible through a web browser interface and application programming interfaces (APIs) enabling integration with third-party EHR systems, practice management systems, clearinghouses, and payer portals.

System 100 supports the following integration modalities:
1. HL7 FHIR Interface: The system accepts and produces healthcare data in Health Level 7 (HL7) Fast Healthcare Interoperability Resources (FHIR) R4 format, enabling standardized data exchange with FHIR-compliant EHR systems and health information exchanges.
2. Direct API Integration: RESTful API endpoints enable third-party practice management systems and EHR platforms to submit encounter data to the AI intake layer 110 and receive validated, priced, and mapped claim data in return.
3. Clearinghouse Integration: The EDI Transaction Generator 160 supports submission of ANSI ASC X12N 837 transactions through healthcare clearinghouses for routing to payers not supporting direct EDI submission.
4. Direct Payer Connectivity: For payers supporting direct EDI submission, the EDI Transaction Generator 160 supports direct secure transmission through HIPAA-compliant secure file transfer protocol (SFTP) and AS2 connections.

### Data Security and HIPAA Compliance

The system 100 processes Protected Health Information (PHI) as defined under 45 C.F.R. § 160.103 and is designed and operated in compliance with the HIPAA Security Rule, 45 C.F.R. § § 164.302-164.318. Security measures implemented in the system 100 include:
1. Encryption at Rest: All PHI stored within the system 100, including encounter data, ABC code records, validated claim data, and audit logs, is encrypted using AES-256 encryption.
2. Encryption in Transit: All PHI transmitted between system components and between the system and external parties is encrypted using TLS 1.2 or higher.
3. Role-Based Access Control: Access to system functions and data is restricted based on user role, with distinct permission sets for clinical coders, compliance reviewers, system administrators, and read-only reporting users.
4. Audit Trail: All system access events, data modifications, validation decisions, and EDI transmissions are logged to an immutable audit trail database as described in Section 5.9.
5. Business Associate Agreements: The system operator maintains executed Business Associate Agreements (BAAs) with all covered entities and business associates accessing the system, as required under 45 C.F.R. § 164.504(e).

### Alternative Embodiments

While the preferred embodiments described herein contemplate a cloud-based SaaS implementation, the invention is not so limited. Alternative embodiments include:

On-Premises Deployment: In embodiments where covered entity security policies or regulatory requirements preclude cloud-based PHI processing, the system 100 may be deployed as an on-premises software installation on covered entity-controlled server infrastructure, with all components operating within the covered entity's network perimeter.
1. Hybrid Deployment: In hybrid embodiments, PHI processing components including the AI intake layer 110, Scope-of-Practice Validation Engine 130, and EDI Transaction Generator 160 operate on covered entity-controlled infrastructure, while non-PHI components including the ABC Code Database 120, jurisdiction-specific regulatory database 132, and Pricing Engine 140 operate as cloud-based services receiving de-identified queries.
2. Direct ABC Code EDI Transmission: In embodiments where ABC codes are formally adopted under future federal rulemaking, the Code Mapping Module 150 may be configured to transmit ABC codes directly in ANSI ASC X12N 837 transactions without translation to HCPCS Level I or Level II codes. However, mapping to HCPCS codes will continue to determine legal use of HCPCS by non-allopathic providers.
3. Standalone Pricing Module: In embodiments where a payer, health system, or government agency requires RVU-based pricing data for non-allopathic procedures independent of the full claim creation workflow, the Pricing Engine 140 may be deployed as a standalone module providing RVU and UPC pricing data through an API without requiring integration with the AI intake layer 110 or EDI transaction generator 160.

What is claimed:

1. A computer-implemented system for processing healthcare transactions for non-allopathic healthcare providers, the system comprising:
a. at least one processor and non-transitory memory storing instructions;
b. a clinical data intake layer configured to receive encounter data;
c. an optional artificial intelligence processing engine configured to: extract one or more candidate healthcare interventions; and generate one or more corresponding procedure codes;
d. a jurisdiction-specific Scope-of-Practice Validation Engine configured to: determine whether a provider is legally authorized to perform each candidate intervention under applicable law;
e. a control module configured to: automatically exclude any intervention determined to be not legally authorized from inclusion in a billable transaction dataset prior to generation of a healthcare claim;
f. a pricing engine configured to assign reimbursement values;
g. a code translation module configured to map validated interventions to one or more standardized healthcare code sets; and
h. a transaction generation module configured to generate a healthcare transaction for electronic submission; wherein the system determines legal permissibility of healthcare interventions prior to claim creation and prevents inclusion of unauthorized interventions in any generated healthcare transaction.

2. The system of claim 1, wherein the system provides a computer-implemented method comprising: receiving encounter data; extracting candidate interventions using an artificial intelligence engine; determining legal authorization based on jurisdiction-specific rules; removing any intervention determined to be unauthorized from a claim dataset prior to claim generation; assigning pricing to authorized interventions; and generating a healthcare transaction including only authorized interventions.

3. The system of claim 1 further wherein the artificial intelligence engine generates candidate procedure codes from clinical input; the system validates each code against jurisdiction-specific legal rules; and the artificial intelligence engine is constrained to output only legally permissible interventions for claim generation.

4. The system of claim 1, further wherein the system is implemented within at least one of: a healthcare payer system, a claims clearinghouse, or an adjudication platform, configured to: receive healthcare claim data; evaluate interventions for legal authorization;
and reject, modify, or remove unauthorized interventions prior to adjudication.

5. The system of claim 1, wherein the system further comprises a legal classification layer configured to map a plurality of standardized procedure codes to a legal intervention classification, and wherein the Scope-of-Practice Validation Engine determines authorization based on the legal intervention classification.

6. The system of claim 1, for use with a regulatory database, further wherein the system continuously updates the regulatory database; and claim eligibility is dynamically modified based on changes in jurisdiction-specific Scope-of-Practice rules.

7. The system of claim 1, wherein the EDI transaction generator is further configured to produce at least one of: ANSI ASC X12N 835 Health Care Claim Payment/Advice transactions; ANSI ASC X12N 276/277 Health Care Claim Status transactions; and ANSI ASC X12N 278 Health Care Services Review transactions.

8. The system of claim 1, wherein the Code Mapping Module is further configured to identify ABC codes having no corresponding HCPCS equivalent and route such codes for unlisted procedure code processing or supplemental documentation generation.

9. The system of claim 1, wherein the ABC Code Database stores, for each ABC code, at least: a unique alphanumeric code identifier; a 5 all-alphabetic character ABC code; a clinical description of the associated intervention; one or more associated non-allopathic provider type designations; one or more applicable geographic jurisdiction flags; an RVU value or UPC cross-reference; and a mapping relationship to a corresponding HCPCS code where available.

10. The system of claim 1, wherein the pricing engine is further configured to assign procedure pricing using a total of methodology incorporating work Relative Value Units, practice expense Relative Value Units, and malpractice Relative Value Units.

11. The system of claim 1, wherein non-allopathic healthcare providers comprise at least one of: acupuncturists, advanced practice registered nurses, behavioral health providers, chiropractors, alternative/holistic/functional medicine doctor, exercise specialist, licensed massage therapists, certified nurse-midwives, naturopathic doctors, occupational therapists, physical therapists, pharmacists, nutritionists, dieticians, herbalists, indigenous medicine and spiritual care providers.

12. A computer-implemented method for processing healthcare transactions for non-allopathic healthcare and alternative/holistic/functional medical providers, the method comprising:
a. receiving, by a processor, encounter data comprising at least one of structured electronic health records, free-text clinical notes, audio transcriptions, SOAP notes, and payer edit data;
b. normalizing, by an artificial intelligence intake layer, the received encounter data regardless of input format or source;
c. extracting, from the normalized encounter data, one or more candidate non-allopathic healthcare interventions;
d. recommending, from an ABC Code Database, one or more ABC codes corresponding to the extracted candidate interventions, and assigning a confidence score to each recommended ABC code;
e. validating, by a Scope-of-Practice validation engine, each recommended ABC code against jurisdiction-specific Scope-of-Practice rules applicable to the provider type and geographic jurisdiction associated with the encounter data;
f. assigning, by a pricing engine, Relative Value Units (RVUs) to validated procedure interventions and cross-referencing Universal Product Codes (UPCs) for validated product interventions;
g. mapping, by a Code Mapping Module, validated and priced ABC codes to Health Care Procedure Coding System (HCPCS) codes mandated under HIPAA Transaction and Code Set standards; and
h. generating, by an EDI transaction generator, ANSI ASC X12N 837 Health Care Claim transactions incorporating the mapped HCPCS codes for electronic transmission to a payer.

13. The method of claim 12, further comprising routing any encounter record having a confidence score below a configurable threshold to a human review interface prior to EDI transmission.

14. The method of claim 12, further comprising removing from the claim workflow any intervention for which the Scope-of-Practice Validation Engine determines the provider is not legally authorized to perform the intervention under applicable jurisdiction-specific law.

15. The method of claim 12, wherein receiving encounter data includes receiving audio transcriptions generated by at least one of a speech recognition system, an ambient listening device, a clinical transcription service, or a large language model documentation workflow.

16. The method of claim 12, further comprising logging all validation decisions, pricing assignments, code mappings, and human review outcomes for audit, compliance reporting, and outcomes analysis purposes.

17. The method of claim 12, wherein validating each recommended ABC code comprises returning one of three validation states: AUTHORIZED, NOT AUTHORIZED, or REVIEW REQUIRED, based on a query to a jurisdiction-specific regulatory database storing current Scope-of-Practice rules for non-allopathic provider types.

18. The method of claim 12, further comprising identifying missing data elements required for complete claim creation and flagging such records before EDI transmission.

19. A non-transitory computer-readable medium storing instructions that, when executed by one or more processors, cause the processors to perform operations comprising:
a. receiving encounter data associated with a non-allopathic healthcare provider encounter;
b. processing the encounter data through an artificial intelligence normalization layer to extract candidate interventions and recommend corresponding ABC codes;
c. validating each recommended ABC code against a jurisdiction-specific scope-of-practice database to confirm the non-allopathic provider's legal authority to perform the corresponding intervention;
d. assigning pricing to validated interventions using Relative Value Units for procedures and Universal Product Codes for products;
e. mapping validated and priced ABC codes to HIPAA-mandated HCPCS codes; and
f. generating a compliant ANSI ASC X12N 837 Health Care Claim transaction for electronic submission to a payer.

20. The non-transitory computer-readable medium of claim 19, wherein the operations further comprise routing encounter records with low-confidence AI recommendations or ambiguous Scope-of-Practice authorization to a human review interface before claim creation is completed.

21. The non-transitory computer-readable medium of claim 19, wherein the artificial intelligence normalization layer is configured to process encounter data received from large language model documentation workflows and validate AI-generated code recommendations against jurisdiction-specific provider Scope-of-Practice rules before inclusion in a healthcare claim transaction.
